(19) United States
(12) Patent Application Publication
(10) Pub. No.: US 2025/0166840 A1
(43) Pub. Date: May 22, 2025

(54) METHOD AND DEVICE FOR DISEASE RISK PREDICTION, STORAGE MEDIUM AND ELECTRONIC DEVICE

(71) Applicant: BOE TECHNOLOGY GROUP CO., LTD., Beijing (CN)

(72) Inventor: Zhenzhong ZHANG, Beijing (CN)

(21) Appl. No.: 19/019,256

(22) Filed: Jan. 13, 2025

Related U.S. Application Data
(63) Continuation-in-part of application No. 17/795,640, filed on Jul. 27, 2022.

(51) Int. Cl. G16H 50/30; G16H 10/60

(52) U.S. Cl. CPC G16H 50/30; G16H 10/60

(57) ABSTRACT

The present disclosure provides a method and device for disease risk prediction, a computer readable storage medium and an electronic device, which relates to the field of data processing. The method includes: obtaining risk feature data of a target user; perform data enhancement on the obtained risk feature data to obtain enhanced risk feature data; and determining, by a disease risk prediction model based on the risk feature data, a disease risk value of the target user and a reliability score of the disease risk value.

### CROSS REFERENCE

The present application is a continuation-in-part application of the U.S. patent application Ser. No. 17/795,640, which is based upon International Application No. PCT/CN2021/096149, filed on May 26, 2021, and the entire contents thereof are incorporated herein by reference.

### TECHNICAL FIELD

The present disclosure relates to the field of data processing, in particular, to a method and device for disease risk prediction, a computer readable storage medium and an electronic device.

### BACKGROUND

In the field of medical technology, it is of great significance to predict the risk of a user's occurrence of a certain disease. For example, accurate risk prediction can realize early discover and early intervention of the disease, thereby slowing the occurrence of the disease.

It should be noted that, information disclosed in the above background portion is provided only for better understanding of the background of the present disclosure, and thus it may contain information that does not form the prior art known by those ordinary skilled in the art.

### SUMMARY

The present disclosure provides a method and device for disease risk prediction, a computer readable storage medium and an electronic device.

The present disclosure provides a method for disease risk prediction, including:
obtaining risk feature data of a target user; and
performing data enhancement on the obtained risk feature data to obtain enhanced risk feature data.

In an exemplary embodiment of the present disclosure, the performing data enhancement on the obtained risk feature data to obtain enhanced risk feature data includes:
inputting the obtained risk feature data to a variational autoencoder (VAE); and
obtaining the enhanced risk feature data from an output of the VAE.

In an exemplary embodiment of the present disclosure, the VAE includes:
an encoder; including an input layer, a hidden layer and an output layer, and configured to output a mean u and logarithmic variance log (σ²) of latent distribution in response to inputting a risk feature data x; and
a decoder, including an input layer, a hidden layer and an output layer, and configured to output a reconstructed value % in response to inputting a latent variable z, wherein the latent variable z is sampled from a latent space according to z=μ+σ×ε, wherein ε follows a standard normal distribution.

In an exemplary embodiment of the present disclosure, the method for disease risk prediction further includes:
obtaining training data and training the VAE using the training data,
wherein a loss function of the training is: VAE_loss=recon_loss+KL, wherein recon_loss=∥\tilde{x}−x∥², and

$$KL = - log σ + \frac{{μ}^{2} + {σ}^{2}}{2} - \frac{1}{2} .$$

In an exemplary embodiment of the present disclosure, the method for disease risk prediction further includes:
determining, by a disease risk prediction model based on the risk feature data, a disease risk value of the target user and a reliability score of the disease risk value.

In an exemplary embodiment of the present disclosure, the enhanced risk feature data includes original copy of the risk feature data and k copies of the output of the VAE obtained by inputting k times the risk feature data to the VAE, wherein k is an integer greater than 1, and
the determining, by the disease risk prediction model based on the enhanced risk feature data, the disease risk value of the target user and the reliability score of the disease risk value includes:
obtaining k+1 output values y̅′̅, y̅₁̅, . . . , y̅ₖ̅ by inputting each copy of the enhanced risk feature data into the disease risk prediction model;
determining the disease risk value of the target user as

$$\overline{y} = \frac{\overline{{y}^{′}} + \overline{{y}_{1}} + … + \overline{{y}_{k}}}{k + 1} ;$$

and
determining the reliability score of the disease risk value as

$$\frac{{( \overline{{y}^{′}} - \overline{y} )}^{2} + {( \overline{{y}_{1}} - \overline{y} )}^{2} + … + {( \overline{{y}_{k}} - \overline{y} )}^{2}}{k + 1} .$$

In an exemplary embodiment of the present disclosure, the determining, by the disease risk prediction model based on the risk feature data, the disease risk value of the target user includes:
the disease risk prediction model including a first risk prediction parameter; and
obtaining the disease risk value of the target user based on the risk feature data and the first risk prediction parameter.

In an exemplary embodiment of the present disclosure, the method includes training the disease risk prediction model to obtain the first risk prediction parameter,
wherein the training the disease risk prediction model to obtain the first risk prediction parameter includes:
inputting feature training data into the disease risk prediction model to determine a second risk prediction parameter;
determining a reliability score of the disease risk prediction model according to the second risk prediction parameter; and
training the disease risk prediction model based on the reliability score to obtain the first risk prediction parameter.

In an exemplary embodiment of the present disclosure, the feature training data includes risk feature training data and disease risk training data, and
the inputting the feature training data into the disease risk prediction model to determine the second risk prediction parameter includes:
determining a mapping relationship between the risk feature training data and the disease risk training data in a first part of the feature training data to establish the disease risk prediction model;
inputting the risk feature training data and disease risk training data in a second part of the feature training data into the disease risk prediction model, and constructing a objective function; and
determining the second risk prediction parameter according to the objective function.

In an exemplary embodiment of the present disclosure, the determining the mapping relationship between the risk feature training data and the disease risk training data in the first part of the feature training data includes:
obtaining a latent factor vector corresponding to the risk feature training data;
obtaining a distribution of the risk feature training data and a distribution of the disease risk training data based on the latent factor vector; and
establishing the mapping relationship between the risk feature training data and the disease risk training data according to the distribution of the risk feature training data and the distribution of the disease risk training data.

In an exemplary embodiment of the present disclosure, the mapping relationship between the risk feature training data and the disease risk training data is:

$$p ( {y}_{n} \vert {X}_{n} ) = \int p ( {y}_{n} , {Z}_{n} \vert {X}_{n} ) {dZ}_{n} = \frac{\int p ( {Z}_{n} ) \times p ( {X}_{n} \vert {Z}_{n} ) \times p ( {y}_{n} \vert {Z}_{n} ) d {Z}_{n}}{\int p ( {Z}_{n} ) \times p ( {X}_{n} \vert {Z}_{n} ) {dZ}_{n}} = N ( {y}_{n} \vert {X}_{n}^{T} v , {W}_{y} C {W}_{y}^{T} + {σ}_{2}^{2} )$$

wherein C=(1+σ₁⁻²Wₓ^{T}Wₓ)⁻¹, v=σ₁⁻²WₓCW_{y}^{T}, Xₙ is the risk feature training data of the n-th user, yₙ is the disease risk training data of the n-th user, Zₙ is the latent factor vector corresponding to the risk feature training data of the n-th user, and Wₓ, W_{y}, σ₁, σ₂ are the second risk prediction parameter in the disease risk prediction model.

In an exemplary embodiment of the present disclosure, the objective function is max ln p(Y|X), and wherein Y is the disease risk training data, X is the risk feature training data, and
the determining the second risk prediction parameter according to the objective function includes:
training the risk feature training data and the disease risk training data in the second part of the feature training data using a maximum likelihood estimation algorithm, and obtaining the second risk prediction parameter at a maximum probability value of the objective function.

In an exemplary embodiment of the present disclosure, the determining the reliability score of the disease risk prediction model according to the second risk prediction parameter includes:
determining a performance parameter corresponding to the second risk prediction parameter in the mapping relationship; and
calculating the performance parameter to obtain the reliability score of the disease risk prediction model.

In an exemplary embodiment of the present disclosure, the performance parameter is W_{y}CW_{y}^{T}+σ₂², wherein, C=(1+σ₁⁻²Wₓ^{T}Wₓ)⁻¹, and Wₓ, W_{y}, σ₁, σ₂ are the second risk prediction parameter in the disease risk prediction model.

In an exemplary embodiment of the present disclosure, the training the disease risk prediction model based on the reliability score to obtain the first risk prediction parameter includes:
acquiring a third part of the feature training data when the reliability score is lower than a preset threshold; and
training the disease risk prediction model based on the third part of the feature training data, to obtain the first risk prediction parameter after the training is completed.

In an exemplary embodiment of the present disclosure, the determining reliability score of the disease risk value by the disease risk prediction model includes:
determining the performance parameter corresponding to the first risk prediction parameter in the mapping relationship; and
calculating the performance parameter to obtain the reliability score for the disease risk value.

In an exemplary embodiment of the present disclosure, the obtaining the disease risk value of the target user based on the risk feature data and the first risk prediction parameter includes:
determining the disease risk value of the target user according to a relationship between the risk feature data and the first risk prediction parameter:

$${y}_{j} = {x}_{j}^{T} {σ}_{I}^{′ - 2} {{W}_{x}^{′} ( I + {σ}_{I}^{′ - 2} {W}_{x}^{′ T} {W}_{x}^{′} )}^{- 1} {W}_{y}^{′ T}$$

wherein xⱼ is the risk feature data of the target user, yⱼ is the disease risk value of the target user, and Wₓ′, W_{y}′, σ₁′, σ₂′ are the first risk prediction parameter in the disease risk prediction model.

The present disclosure provides a device for disease risk prediction, including:
a data obtaining module, configured to obtain risk feature data of a target user; and
a data enhancement module, configured to perform data enhancement on the obtained risk feature data to obtain enhanced risk feature data.

In an exemplary embodiment of the present disclosure, the device for disease risk prediction further includes:
a data determining module, configured to determine, by a disease risk prediction model based on the risk feature data, a disease risk value of the target user and a reliability score of the disease risk value.

In an exemplary embodiment of the present disclosure, the device further includes:
a data output module, configured to output the disease risk value of the target user and the reliability score of the disease risk value to a terminal device and present the disease risk value of the target user and the reliability score of the disease risk value to the user.

The present disclosure provides a computer-readable storage medium stored thereon with a computer program, which when being executed by a processor, implement any of the above method.

The present disclosure provides an electronic device, including a processor; and a memory, configured to store instructions executable by the processor, wherein the processor is configured to implement any of the above method.

It is to be understood that both the foregoing general description and the following detailed description are exemplary and explanatory only and are not restrictive of the invention.

### BRIEF DESCRIPTION OF THE DRAWINGS

The accompanying drawings, which are incorporated in and constitute a part of this specification, illustrate embodiments consistent with the disclosure and together with the description serve to explain the principles of the disclosure. Obviously, the drawings in the following description are only some embodiments of the present disclosure, and for those of ordinary skill in the art, other drawings can also be obtained from these drawings without creative effort.

FIG. 1 shows a schematic diagram of an exemplary system architecture of a disease risk prediction method and device to which embodiments of the present disclosure can be applied;

FIG. 2 shows a schematic structural diagram of a computer system suitable for implementing an electronic device according to an embodiment of the present disclosure;

FIG. 3A schematically shows a flowchart of a disease risk prediction method according to an embodiment of the present disclosure;

FIG. 3B schematically shows a flowchart of a disease risk prediction method according to an embodiment of the present disclosure;

FIG. 4 schematically shows a flowchart of determining a first risk prediction parameter according to an embodiment of the present disclosure;

FIG. 5 schematically shows a flowchart of determining a second risk prediction parameter according to an embodiment of the present disclosure;

FIG. 6 schematically shows a flowchart of modeling the disease prediction model according to a specific embodiment of the present disclosure;

FIG. 7A schematically shows a schematic diagram of the disease prediction model according to a specific embodiment of the present disclosure;

FIG. 7B schematically shows a schematic diagram of the VAE according to a specific embodiment of the present disclosure;

FIG. 8A schematically shows a block diagram of a disease risk prediction device according to an embodiment of the present disclosure; and

FIG. 8B schematically shows a block diagram of a disease risk prediction device according to an embodiment of the present disclosure.

### DETAILED DESCRIPTION

Example embodiments will now be described more fully with reference to the accompanying drawings. Example embodiments, however, can be embodied in various forms and should not be construed as limited to the examples set forth herein; rather, these embodiments are provided so that this disclosure will be thorough and complete, and will fully convey the concept of example embodiments to those skilled in the art. The described features, structures, or characteristics may be combined in any suitable manner in one or more embodiments. In the following description, numerous specific details are provided in order to give a thorough understanding of the embodiments of the present disclosure. However, those skilled in the art will appreciate that the technical solutions of the present disclosure may be practiced without one or more of the specific details, or other methods, components, devices, steps, and the like may be employed. In other instances, well-known solutions have not been shown or described in detail to avoid obscuring aspects of the present disclosure.

Furthermore, the drawings are merely schematic illustrations of the present disclosure and are not necessarily drawn to scale. The same reference numerals in the drawings denote the same or similar parts, and thus their repeated descriptions will be omitted. Some of the block diagrams shown in the figures are functional entities, which do not necessarily correspond to physically or logically separate entities. These functional entities may be implemented in software, or in one or more hardware modules or integrated circuits, or in different networks and/or processor devices and/or microcontroller devices.

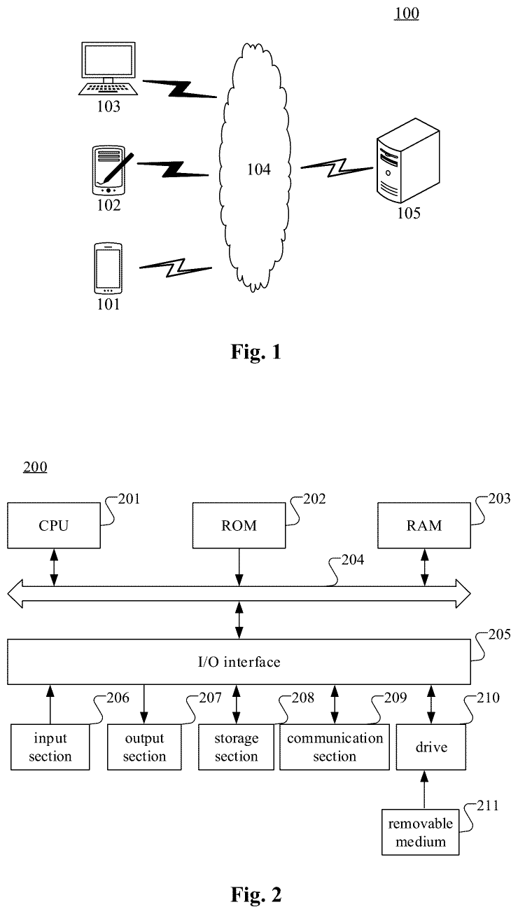

FIG. 1 shows a schematic diagram of an exemplary system architecture of a disease risk prediction method and device to which embodiments of the present disclosure can be applied.

As shown in FIG. 1, the system architecture 100 may include one or more of terminal devices 101, 102, 103, a network 104 and a server 105. The network 104 is a medium used to provide a communication link between the terminal devices 101, 102, 103 and the server 105. The network 104 may include various connection types, such as wired, wireless communication links, or fiber optic cables, and the like. The terminal devices 101, 102, and 103 may be various electronic devices, including but not limited to desktop computers, portable computers, smart phones, and tablet computers. It should be understood that the numbers of terminal devices, networks and servers in FIG. 1 are only illustrative. There can be any number of terminal devices, networks, and servers according to implementation needs. For example, the server 105 may be a server cluster composed of multiple servers, or the like.

The disease risk prediction method provided by the embodiment of the present disclosure is generally executed by the server 105. Correspondingly, the disease risk prediction device is generally set in the server 105. After the execution of the server, the prediction result can be sent to the terminal device, and the terminal device can present the result to the user. However, those skilled in the art can easily understand that the disease risk prediction method provided by the embodiments of the present disclosure can also be executed by one or more of the terminal devices 101, 102, and 103. Correspondingly, the disease risk prediction device can also be set in the terminal devices 101, 102, and 103, for example, after being executed by the terminal device, the prediction result may be directly displayed on the display screen of the terminal device, or the prediction result may be provided to the user by means of voice broadcast. There is no special restriction on this in the exemplary embodiment.

FIG. 2 shows a schematic structural diagram of a computer system suitable for implementing an electronic device according to an embodiment of the present disclosure;

It should be noted that the computer system 200 of the electronic device shown in FIG. 2 is only an example, and should not impose any limitations on the functions and scope of use of the embodiments of the present disclosure.

As shown in FIG. 2, the computer system 200 includes a central processing unit (CPU) 201 that can perform various appropriate actions and processes according to programs stored in a read only memory (ROM) 202 or a program loaded into a random access memory (RAM) 203 from a storage section 208. In the RAM 203, various programs and data necessary for system operation are also stored. The CPU 201, the ROM 202, and the RAM 203 are connected to each other through a bus 204. An input/output (I/O) interface 205 is also connected to the bus 204.

The following components are connected to the I/O interface 205: an input section 206 including a keyboard, a mouse, etc.; an output section 207 including a cathode ray tube (CRT), a liquid crystal display (LCD), etc., and a speaker, etc.; a storage section 208 including a hard disk, etc.; and a communication section 209 including a network interface card such as a LAN card, a modem, and the like. The communication section 209 performs communication processing via a network such as the Internet. A drive 210 is also connected to the I/O interface 205 as needed. A removable medium 211, such as a magnetic disk, an optical disk, a magneto-optical disk, a semiconductor memory, etc., is mounted on the drive 210 as needed so that a computer program read therefrom is installed into the storage section 208 as needed.

In some embodiments, the disease risk prediction methods described in this disclosure are performed by a processor of an electronic device. In some embodiments, the risk feature data of the target user obtained according to the expert knowledge, as well as the risk feature training data and the disease risk training data for constructing and training the disease risk prediction model, are input through the input section 206. For example, the target user's risk feature data, risk feature training data and disease risk training data and other information are input by the user interface of the electronic device. In some embodiments, the output part 207 outputs information such as the disease risk value of the target user and the reliability score corresponding to the disease risk value.

In particular, according to embodiments of the present disclosure, the processes described below with reference to the flowcharts may be implemented as computer software programs. For example, embodiments of the present disclosure include a computer program product including a computer program carried on a computer-readable medium, the computer program containing program code for performing the method illustrated in the flowchart. In such an embodiment, the computer program may be downloaded and installed from the network via the communication portion 209 and/or installed from the removable medium 211. When the computer program is executed by the central processing unit (CPU) 201, various functions defined in the method and apparatus of the present application are performed.

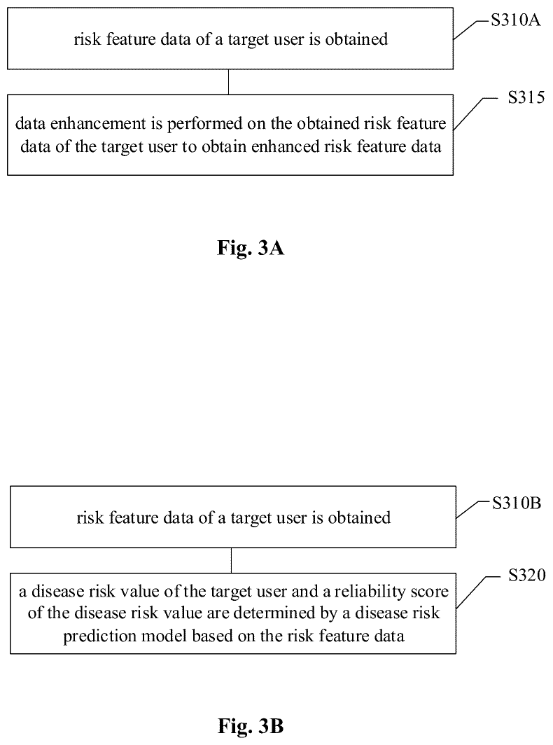

As another aspect, the present application also provides a computer-readable medium. The computer-readable medium may be included in the electronic device described in the above embodiments. It may also exist alone without being assembled into the electronic device. The above-mentioned computer-readable medium carries one or more programs, and when the above-mentioned one or more programs are executed by an electronic device, causes the electronic device to implement the methods described in the following embodiments. For example, the electronic device can implement various steps as shown in FIG. 3A to FIG. 6.

It should be noted that the computer-readable medium shown in the present disclosure may be a computer-readable signal medium or a computer-readable storage medium, or any combination of the above two. The computer readable storage medium can be, for example, but not limited to, an electrical, magnetic, optical, electromagnetic, infrared, or semiconductor system, apparatus or device, or a combination of any of the above. More specific examples of computer readable storage media may include, but are not limited to, electrical connections with one or more wires, portable computer disks, hard disks, random access memory (RAM), read only memory (ROM), erasable Programmable read only memory (EPROM or flash memory), optical fiber, portable compact disk read only memory (CD-ROM), optical storage devices, magnetic storage devices, or any suitable combination of the above. In the present disclosure, a computer-readable storage medium may be any tangible medium that contains or stores a program that can be used by or in conjunction with an instruction execution system, apparatus, or device. In the present disclosure, however, a computer-readable signal medium may include a data signal propagated in baseband or as part of a carrier wave, carrying computer-readable program code therein. Such propagated data signals may take a variety of forms, including but not limited to electromagnetic signals, optical signals, or any suitable combination of the foregoing. A computer-readable signal medium can also be any computer-readable medium other than a computer-readable storage medium that can transmit, propagate, or transport the program for use by or in connection with the instruction execution system, apparatus, or device. Program code embodied on a computer readable medium may be transmitted using any suitable medium including, but not limited to, wireless, wireline, optical fiber cable, RF, etc., or any suitable combination of the foregoing.

The technical solutions of the embodiments of the present disclosure are described in detail below.

In the exemplary embodiment of the present disclosure, gestational diabetes risk prediction can be used as an example for illustration. Gestational diabetes occurs during pregnancy, and its incidence has increased significantly in recent years. At present, gestational diabetes has become one of the most common complications of pregnancy. It is important to note that women with gestational diabetes are also at increased risk of developing postpartum diabetes. Therefore, accurate risk prediction of gestational diabetes to achieve early discover and early intervention of the disease has important clinical significance in slowing the occurrence and development of complications.

At present, for a more commonly used risk prediction model, the Logistic Regression (LR) model, the LR model can use a linear function to model the posterior probability of the class label, and directly output the normalized probability with an interval of 0 to 1. However, in the LR model, the premise of modeling is to assume that each risk factor is independent, but in fact some risk factors are related, for example, in the modeling process of the LR model, it is assumed that the body height and body weight does not affect each other, but in fact body height and body weight are not independent of each other. Generally, people with higher body height will be heavier in body weight. Therefore, ignoring the associations between individual risk factors may reduce the accuracy of disease risk prediction. At the same time, after using the LR model for disease risk prediction, the reliability of the prediction model cannot be given. The reliability is a key factor to measure the accuracy of the risk prediction model. The higher the reliability, the more credible the results of the risk prediction are. It should be noted that the disease types to which the disease risk prediction method in the examples of the present disclosure is applicable include but are not limited to gestational diabetes, which is not specifically limited in the present disclosure.

Based on one or more of the above problems, this example embodiment provides a disease risk prediction method, which can be applied to the above-mentioned server 105 or one or more of the above-mentioned terminal devices 101, 102, and 103, which is not particularly limited in the exemplary embodiment. Referring to FIG. 3A, the disease risk prediction method may include the following steps S310A and S315.

In step S310A, risk feature data of a target user is obtained.

In step S315, data enhancement is performed on the obtained risk feature data to obtain enhanced risk feature data. The enhanced reis feature data may be used for the disease risk prediction.

In the embodiment, the data enhancement may be performed by using a variational autoencoder (VAE). Accordingly, the method may include inputting the obtained risk feature data to a variational autoencoder (VAE); and obtaining the enhanced risk feature data from an output of the VAE.

In further detail, the VAE may include an encoder; including an input layer, a hidden layer and an output layer, and configured to output a mean u and logarithmic variance log(σ²) of latent distribution in response to inputting a risk feature data x; and a decoder, including an input layer, a hidden layer and an output layer, and configured to output a reconstructed value \tilde{x} in response to inputting a latent variable z, wherein the latent variable z is sampled from a latent space according to z=μ+σ×ε, wherein & follows a standard normal distribution.

In an embodiment of the present disclosure, the VAE may be trained before using the VAE to obtain the enhanced risk feature data. In particular, training data for the VAE may be firstly obtained, and the VAE may then be trained using the training data. The loss function of the training may be VAE_loss=recon_loss+KL, wherein recon_loss=∥\tilde{x}−x∥, and

$$KL = - log σ + \frac{{μ}^{2} + {σ}^{2}}{2} - \frac{1}{2} .$$

In the embodiment, the recon_loss may represent the reconstruction loss, which is the mean square error (MSE) to measure the difference between the decoder output \tilde{x} and the true input data x, and the KL may represent the divergence, which is used to calculate the difference between the potential distribution and the standard normal distribution.

In an embodiment of the present disclosure, referring to FIG. 3B, the disease risk prediction method may include the following steps S310B and S320.

In step S310B, risk feature data of a target user is obtained.

In step S320, a disease risk value of the target user and a reliability score of the disease risk value are determined by a disease risk prediction model based on the risk feature data.

In the present embodiment, the risk feature data obtained in the step S310B may be the enhanced risk feature data obtained in the step S315 of FIG. 3A. In other embodiments, the risk feature data obtained in the step S310B may also be other risk feature data, which is not limited in the present application.

In one embodiment, the enhanced risk feature data obtained in the step S315 of FIG. 3A is used as the risk feature data obtained in the step S310B, wherein the enhanced risk feature data includes original copy of the risk feature data and k copies of the output of the VAE obtained by inputting k times the risk feature data to the VAE, wherein k is an integer greater than 1. That is, the risk value x is input to the VAE for k times, and the enhanced risk feature data includes the risk value x, and all the output of the VAE of k inputs. In this embodiment, all the k+1 pieces of the enhanced risk feature data may be input to the disease risk prediction model respectively, and thus k+1 output values y̅′̅, y̅₁̅, . . . , y̅ₖ̅ may be obtained. After obtaining the k+1 output values y̅′̅, y̅₁̅, . . . , y̅ₖ̅, the disease risk value of the target user corresponding to the risk feature data x may be represented as

$$\overline{y} = \frac{\overline{{y}^{′}} + \overline{{y}_{1}} + … + \overline{{y}_{k}}}{k + 1} ;$$

and the reliability score of the disease risk value may be represented as

$$\frac{{( \overline{{y}^{′}} - \overline{y} )}^{2} + {( \overline{{y}_{1}} - \overline{y} )}^{2} + … + {( \overline{{y}_{k}} - \overline{y} )}^{2}}{k + 1} .$$

In the disease risk prediction method provided by the exemplary embodiment of the present disclosure, by obtaining the risk feature data of the target user, a disease risk value of the target user and a reliability score of the disease risk value are determined by a disease risk prediction model based on the risk feature data. The method can more accurately determine the disease risk of the target user through the disease risk prediction model, and can obtain the reliability of the disease risk prediction model.

Hereinafter, the above steps of this exemplary embodiment will be described in more detail.

In step S310B, risk feature data of a target user is obtained.

In this exemplary embodiment, the target user may be a patient with a condition related to the disease to be predicted, or a healthy patient undergoing routine disease investigation. The risk feature data may include physical sign data, inspection data, and the like. In some embodiments, the risk feature data corresponding to different diseases may be different, that is, the corresponding risk feature data to be collected may be determined according to the disease to be predicted. For example, when predicting diabetes risk, the corresponding risk feature data can be factors such as weight, family origin, blood pressure, etc. When predicting the risk of cardiovascular and cerebrovascular diseases, the corresponding risk feature data can be waist circumference, total cholesterol content, blood pressure, smoking history and other factors.

To obtain the risk feature data of the target user, the current risk feature data of the target user can be obtained, such as collecting the risk feature data of the target user on the day of disease risk prediction, or the historical risk feature data of the target user can be obtained, such as obtaining the historical risk feature data of the target user one month ago, and the disease risk prediction is carried out based on the acquired historical risk feature data. Exemplarily, the physical examination results of the target user's physical examination in a hospital one month ago can be obtained, which can include physical data such as body height and body weight, as well as inspection data such as blood pressure, blood lipids, and cholesterol, and can also include feature data related to certain diseases.

In this example, when predicting the risk of gestational diabetes for a target user, the risk feature data corresponding to the target user can be obtained. For example, the basic data of the target user can be obtained from the information system of the hospital, and the basic data can include all the risk feature data of the target user, such as the physical sign data of the target user, examination data and feature data related to gestational diabetes, Such as pregnancy, gestational age and other information.

After acquiring the basic data of the target user, data cleaning can be performed on all the risk feature data contained therein. Exemplarily, when the data is incomplete, the corresponding feature attribute can be removed. For example, if the age of the target user is not recorded in the age attribute of the risk feature data, it can be supplemented by deduction from other data. For example, the ID number is used to estimate the age of the target user. If the age of the target user cannot be obtained, this attribute can be removed. For another example, when the data is duplicated, the risk feature data can be deduplicated.

After the data cleaning is completed, feature selection can be performed on the risk feature data obtained by cleaning. Exemplarily, an expert can select risk feature data that is highly related to gestational diabetes based on professional knowledge, or can obtain risk feature data that is highly related to gestational diabetes by matching with the corresponding data in the expert knowledge base. The risk feature data that is less related to gestational diabetes can be removed, to finally obtain risk feature data that can be used for disease risk prediction.

In this example, the risk feature data obtained through feature selection can be sorted according to their relevance to gestational diabetes, such as in descending order, and the top-ranked risk feature data can be used as the risk feature data for disease risk prediction. Exemplarily, the top 11 risk feature data with high correlation with gestational diabetes may be selected according to expert knowledge, which can be particularly referred to Table 1.

TABLE 1

| | | | | | | Normal |
|---|---|---|---|---|---|---|
| No. | Feature ID | Feature name | Data type | Value | Unit | value |
| 1 | birthDate | Age | Integer | | | 35 |
| 2 | weight | Body weight | Integer | | kg | 69 |
| 3 | height | Body height | Integer | | cm | 164 |
| 4 | pregnancy | Pregnant or not | Boolean | Y/N | | Y |
| | | | value | | | |
| 5 | gesweeks | Gestational | Integer | | | 11-13 |
| | | week | | | | |
| 6 | gdmhistory | Gestational | Category | Never given birth/ | | |
| | | diabetes history | | given birth without | | |
| | | | | having gestational | | |
| | | | | diabetes/had | | |
| | | | | gestational diabetes | | |
| 7 | prebirthweight | Weight of the | Integer | | kg | 4 |
| | | last baby at | | | | |
| | | birth | | | | |
| 8 | dmrelative1 | Whether a 1^{st}- | Boolean | Y/N | | N |
| | | degree relative | value | | | |
| | | has diabetes | | | | |
| 9 | dbrelative2 | Whether a 2^{nd}- | Boolean | Y/N | | N |
| | | degree relative | value | | | |
| | | has diabetes | | | | |
| 10 | ovulation | Ovulation | Boolean | Y/N | | N |
| | | drugs | value | | | |
| 11 | racial | Racial origin | Category | East Asian/Afro- | | |
| | | | | Caribbean/South | | |
| | | | | Asian | | |

Table 1 shows 11 pieces of risk feature data that are highly related to gestational diabetes. The feature IDs are: birthDate, weight, height, pregnancy, gesweeks, gdmhistory, prebirthweight, dmrelative1, dbrelative2, ovulation and racial, and the corresponding feature names They are: age, body weight, body height, pregnant or not, gestational week, history of gestational diabetes, weight of the last baby at birth, whether first-degree relatives have diabetes (first-degree relatives refer to the user's parents), whether second-degree relatives have diabetes (Second-degree relatives refer to the user's grandparents and maternal grandparents), ovulation drugs, and racial origin. The data types of pregnant or not, whether first-degree relatives have diabetes, and whether second-degree relatives have diabetes are Boolean values, which can include two values of yes or no. For example, if the target user is pregnant, the Boolean value corresponding to the feature “pregnant or not” is “Y”. The data types of gestational diabetes history and racial origin are categories. Specifically, the feature “gestational diabetes history” can include three categories of features, namely, never given birth, given birth without having gestational diabetes, and had gestational diabetes. The feature “racial origin” may also include three categories of features, namely, East Asian, Afro-Caribbean, and South Asian. In addition, experts can label the user's risk of disease according to the normal value of each risk feature data. For example, the closer the user's risk feature data is to the normal value, the lower the risk of the user's disease.

In step S320, a disease risk value of the target user and a reliability score of the disease risk value are determined by a disease risk prediction model based on the risk feature data.

After obtaining the risk feature data of the target user, a disease risk prediction model can be used to determine the risk value of the target user for gestational diabetes. In the disease risk prediction model, the training data set can be used to learn the mapping relationship between input (such as risk feature data) and output (such as disease risk value), so as to predict the most likely output value corresponding to the new input value. The mapping relationship between the input and the output can be determined by regression, that is to say, the training data is obtained by a function defined by a parameter W. Therefore, the parameter W can be determined according to the training data, so that when a new input value is given, the corresponding output value can be obtained. The disease risk prediction model may include a first risk prediction parameter, and the first risk prediction parameter may be the parameter used in the disease risk prediction model to define a mapping relationship between an input (i.e., the risk feature data) and an output (i.e., the disease risk value).

In this example, disease risk prediction can be performed more accurately by obtaining the correlation between each risk feature data. For example, the disease risk prediction model can be a regression model based on a Gaussian distribution. Specifically, the joint probability density of the training data set can be obtained from the assumed noise distribution, and the regression model can be obtained by finding parameters that maximize the density.

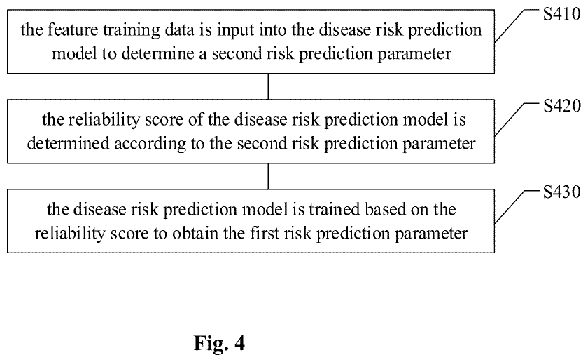

In an example implementation, referring to FIG. 4, the first risk prediction parameter may be determined according to steps S410 to S430, and specifically, the disease risk prediction model may be trained to obtain the first risk prediction parameter.

In order to model the regression model, the basic data of multiple users can be obtained as training data. Similarly, the basic data can include all risk feature data of users. After data cleaning and feature selection are performed on the basic data of multiple users, the feature training data can be obtained. That is, the risk feature data that can be used for modeling can be obtained. For example, as shown in Table 1, 11 risk feature data with high correlation with gestational diabetes can be obtained. It should be noted that the basic data of multiple users may also include disease risk data of the users, that is, the risk of gestational diabetes. The risk of disease can be marked by experts through professional knowledge for each user. For example, the risk of disease can be any value in the interval [0, 10]. Exemplarily, when the risk of disease of the user is 5, it can be expressed that the probability that the user will have gestational diabetes is 50%. Similarly, the risk of disease can also use a value in the interval [0, 1] to represent the probability of the user suffering from gestational diabetes. It can be understood that risk feature data and corresponding disease risk data of any number of users can be obtained, and used as training data, the disease risk prediction model can be trained multiple times to improve the performance of the disease risk prediction model.

In step S410, the feature training data is input into the disease risk prediction model to determine a second risk prediction parameter.

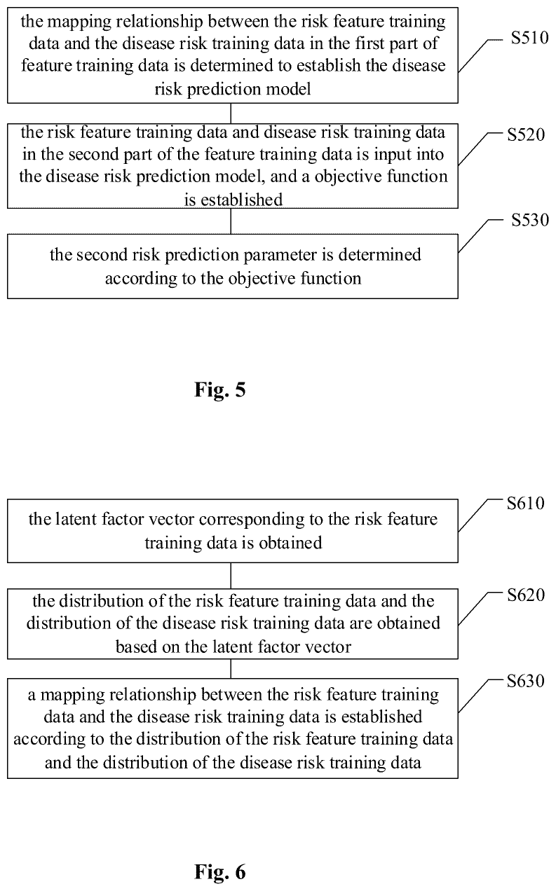

Exemplarily, the risk feature data and disease risk data of “m” users may be obtained, and a regression model may be obtained by modeling the risk feature data and disease risk data of the “m” users, and the second risk prediction parameter may include parameters in the regression model used to define the mapping relationship between input (i.e., risk feature data) and output (i.e., risk value of disease). Specifically, referring to FIG. 5, the second risk prediction parameter may be determined according to steps S510 to S530.

In step S510, the mapping relationship between the risk feature training data and the disease risk training data in the first part of feature training data is determined to establish the disease risk prediction model.

In an example implementation, the risk feature data and disease risk data of “n” users may be selected from the “m” users as the first part of feature training data for establishing the disease risk prediction model. Exemplarily, the risk feature data for the n-th user may include age/35, body weight/69 kg, body height/164 cm, pregnant or not/yes, gestational week/12, gestational diabetes history/, weight of the last baby at birth/4 kg, whether a 1st-degree relative has diabetes/no, whether a 2nd-degree relative has diabetes/no, ovulation drugs/no, racial origin/East Asians, a total of 11 risk factors. According to the 11 risk factors, the expert makes the risk value of the user for gestational diabetes as 1, indicating that the n-th user will have a 10% probability of having gestational diabetes.

Referring to FIG. 6, a disease risk prediction model can be obtained by modeling according to steps S610 to S630.

In step S610, the latent factor vector corresponding to the risk feature training data is obtained.

After obtaining the 11 risk factors of the n-th user, the risk feature matrix Xₙ corresponding to the 11 risk factors can be generated, Xₙ can be an 11×1 matrix, yₙ is the risk of the nth user, and yₙ∈[0, 10]. When generating the risk feature matrix Xₙ, since the 11 risk factors also include features of boolean value type and category type, the features of the two data types can be converted into features of numerical type through One-Hot encoding. One-Hot encoding is also known as one-bit valid encoding. The method is to use an N-bit state register to encode N states, each state has an independent register bit, and at any time, only one bit in the register is valid. For example, the 3 categories of features in the feature “Gestational Diabetes History” can be coded as 1, 2, and 3, respectively, for the features of never given birth, given birth without having gestational diabetes, and had gestational diabetes. Then the category feature corresponding to the target user can be mapped. When the category feature is “never given birth”, it is 1 after mapping, and other category features are 0. After all 11 risk factors are converted into numerical features, the risk factors of each user can also be converted into vectors through the Word Embedding algorithm, such as Word2vec algorithm, Glove algorithm, etc.

In this example, in order to more accurately predict the risk of gestational diabetes, the relationship between each of the 11 risk factors is required to be determined. There may be obvious associations between risk factors, and there may also be potential associations. Such as age and body weight, generally the older the age, the larger the body weight, and the relationship between the two is more obvious. For body height and gestational diabetes history, the relationship between the two cannot be obtained intuitively. Exemplarily, the relationship between risk factors in the Xₙ can be obtained through a latent factor vector, where the latent factor vector is a vector including unobservable random variables.

For example, the latent factor vector corresponding to the n-th user is Zₙ, which may be a new vector obtained by compressing the risk feature matrix Xₙ into a new vector space. Specifically, the latent factor vector Zₙ can be obtained by cross-coding the 11 risk factors of the risk feature matrix Xₙ, that is, the features in Zₙ can be obtained by any combination of 11 risk factors, and the dimension of Zₙ can be a dimension much smaller than the 11 dimensions, for example it can be 5 dimensions, that is, Zₙ can be a 5×1 matrix.

In this example, the risk of disease of the target user can be predicted by the reconstructed low-dimensional matrix Zₙ. Exemplarily, it can be assumed that the Gaussian distribution obeyed by Zₙ is:

$$p ( {Z}_{n} ) = N ( {Z}_{n} \vert 0 , {I}_{L} ) \qquad ( 1 )$$

wherein I_{L} is a 5×5 unit matrix. In order to simplify the calculation, it can be assumed that the initial mean distribution of Zₙ is 0.

In step S620, the distribution of the risk feature training data and the distribution of the disease risk training data are obtained based on the latent factor vector.

When Zₙ is given, the Gaussian distribution obeyed by Xₙ is:

$$p ( {X}_{n} \vert {Z}_{n} ) = N ( {X}_{n} \vert {W}_{x} {Z}_{n} , {σ}_{1}^{2} {I}_{x} ) \qquad ( 2 )$$

wherein p(Xₙ|Zₙ) is the relationship between the risk factors in the Xₙ according to the latent factor vector. Wherein the Iₓ is a 11×11 unit matrix, W is a 11×5 parameter matrix. Based on the latent factor vector Zₙ, Xₙ can be calculated through Wₓ, σ₁²Iₓ is a covariance matrix, and σ₁ is the variance parameter.

When Zₙ is given, the Gaussian distribution obeyed by yₙ is:

$$p ( {y}_{n} \vert {Z}_{n} ) = N ( {y}_{n} \vert {W}_{y} {Z}_{n} , {σ}_{2}^{2} ) \qquad ( 3 )$$

wherein W_{y} is a 11×5 parameter matrix. Based on the latent factor vector Zₙ, yₙ can be calculated through W_{y}, and σ₂ is the variance parameter.

In step S630, a mapping relationship between the risk feature training data and the disease risk training data is established according to the distribution of the risk feature training data and the distribution of the disease risk training data.

After obtaining the distribution p(Xₙ|Zₙ) of the risk feature training data Xₙ and the distribution p(yₙ|Zₙ) of the disease risk training data yₙ, when Xₙ is given, the Gaussian distribution obeyed by yₙ can be obtained as:

$$p ( {y}_{n} \vert {X}_{n} ) = \int p ( {y}_{n} , {Z}_{n} \vert {X}_{n} ) {dZ}_{n} = \frac{\int p ( {Z}_{n} ) \times p ( {X}_{n} \vert {Z}_{n} ) \times p ( {y}_{n} \vert {Z}_{n} ) d {Z}_{n}}{\int p ( {Z}_{n} ) \times p ( {X}_{n} \vert {Z}_{n} ) {dZ}_{n}} = N ( {y}_{n} \vert {X}_{n}^{T} v , {W}_{y} C {W}_{y}^{T} + {σ}_{2}^{2} ) \qquad ( 4 )$$

wherein I is a 5×5 unit matrix.

$$C = {( I + {σ}_{1}^{- 2} {W}_{x}^{T} {W}_{x} )}^{- 1} \qquad ( 5 )$$

$$v = {σ}_{1}^{- 2} {W}_{x} C {W}_{y}^{T} \qquad ( 6 )$$

The p(yₙ|Xₙ) is the mapping relationship between the risk feature training data and the disease risk training data. The mapping relationship is obtained based on the actual relationship between the risk factors, so that the mapping relationship can be more accurate to characterize the relationship between the user's risk feature data and disease risk data. In addition, a regression model can be established through the mapping relationship, and a large amount of sample information can be used for training, so as to facilitate subsequent disease risk prediction.

In step S520, the risk feature training data and disease risk training data in the second part of the feature training data is input into the disease risk prediction model, and a objective function is established.

In an exemplary implementation, the risk feature data and disease risk data of “N” users may be selected from the “m” users as the second part of feature training data for training the disease risk prediction model. The “N” users may include the above-mentioned “n” users, or may be other users excluding the “n” users. The training set corresponding to the “N” users may be:

$$\{ ( {x}_{1} , {y}_{1} ) , … , ( {x}_{i} , {y}_{i} ) , … ( {x}_{N} , {y}_{N} ) \}$$

Taking the risk feature data of each user as the input, and taking the corresponding disease risk data (probability of disease risk) of each user as the output, the regression model is trained to obtain the maximum probability value of the training data.

In the training process, the objective function needs to be constructed first. The objective function can also be called the loss function, which is the performance function in the disease risk prediction model and the key parameter for compiling the model. For example, each training parameter Wₓ, W_{y}, σ₁, σ₂ can be determined by the maximum likelihood algorithm. Specifically, the model parameters can be evaluated according to the given observation data, by performing several experiments and observing the result, it is possible to obtain certain parameter that maximizes the probability of a sample occurrence. In the maximum likelihood algorithm, the corresponding objective function can be:

$$max ln p ( Y \vert X ) = max {\sum }_{i = 1}^{n} ln p ( {y}_{i} \vert {x}_{i} ) \qquad ( 7 )$$

wherein, Y is the disease risk training data, X is the risk feature training data, yᵢ is the risk feature data of each user among the “N” users, and xᵢ is the disease risk data of the each user.

In step S530, the second risk prediction parameter is determined according to the objective function.

The objective function can be used to measure the degree of inconsistency between the predicted value of the model and the true value. Exemplarily, when using the maximum likelihood estimation algorithm to train the risk feature training data xᵢ and the disease risk training data yᵢ in the second part of the feature training data, the risk feature training data xᵢ can be used as the input of the regression model, and the regression model is updated according to the objective function to output the risk of disease training data yᵢ. In the process of updating the regression model according to the objective function, the gradient descent method can be used to continuously calculate the objective function according to the principle of back propagation, and update the parameters in the regression model according to the objective function. When the value of the objective function is the largest, it means that the probability of the occurrence of the training data set is the largest, and the parameters Wₓ, W_{y}, σ₁, σ₂ in the corresponding regression model at this time may be the second risk prediction parameter. In other examples, the parameters can also be optimized by alternating least squares.

In step S420, the reliability score of the disease risk prediction model is determined according to the second risk prediction parameter.

After the second risk prediction parameter Wₓ, W_{y}, σ₁, σ₂ is determined, the performance parameter in the mapping relationship can be determined according to the multiple parameters, that is, the variance parameter W_{y}CW_{y}^{T}+σ₂² in p(yₙ|Xₙ), wherein C=(1+σ₁⁻²Wₓ^{T}Wₓ)⁻¹. The variance parameter can be used to characterize the degree of dispersion between the predicted values, that is, the difference between each output result of the model and the output expectation. In this example, the variance parameter W_{y}CW_{y}^{T}+σ₂² can be used to estimate the reliability of the disease risk prediction model. The larger the variance, the lower the reliability of the disease risk prediction model. After the value of the variance parameter is calculated, the mapping relationship between the variance and the reliability of the disease risk prediction model can be established. For example, the variance is negatively correlated with the reliability of the disease risk prediction model, the value interval of the variance can be [0, 1], and the score interval of the reliability can be [0, 100]. Exemplarily, when the variance is 0.4, the reliability score of the corresponding disease risk prediction model is 60 points, and when the variance is 0.15, the reliability score of the corresponding disease risk prediction model is 85 points. It should be noted that the reliability score of the disease risk prediction model is consistent with the reliability score of the user's disease risk value obtained from the prediction model.

In step S430, the disease risk prediction model is trained based on the reliability score to obtain the first risk prediction parameter.

When the reliability score is lower than the preset threshold, for example, when the reliability score is less than 85 points, the training data can be increased, the model can be retrained by adjusting the number of parameters, and then the model effect can be adjusted. Specifically, the third part of feature training data may be obtained, for example, risk feature data and disease risk data of “M” users may be selected from the “m” users as the third part of training data. The regression model is trained by combining the third part of the feature training data with the second part of the feature training data, and after the training is completed, the reliability of the disease risk prediction model can be estimated according to the optimized risk prediction parameters. For example, the corresponding variance parameter W_{y}CW_{y}^{T}+σ₂² can be calculated, and according to the calculation result, it can be judged whether the reliability score of the corresponding disease risk prediction model is greater than 85 points. If the reliability score is still less than 85 points, the training data can continue to be added to optimize the parameters of the disease risk prediction model. If the reliability score is greater than 85 points, the model parameters obtained by training may be the first risk prediction parameter Wₓ′, W_{y}′, σ₁′, σ₂′. In other examples, the model can also be retrained by increasing the number of iterations, and a better optimization function can be selected to improve the model performance, which is not specifically limited in this example.

After the first risk prediction parameter is obtained, the disease risk value of the target user may be obtained based on the risk feature data and the first risk prediction parameter.

After obtaining the risk feature data xⱼ of the target user, the disease risk value of the target user can be obtained according to the mean vector in the trained disease risk prediction model. The mean vector is:

$${y}_{n} = {x}_{n}^{T} v \qquad ( 8 )$$

wherein v=σ₁⁻²WₓCW_{y}^{T}, and C=(1+σ₁⁻²Wₓ^{T}Wₓ)⁻¹.

It can be seen that when the risk feature data xⱼ of the target user is input into the optimized disease risk prediction model, the first risk prediction parameter of the model is Wₓ′, W_{y}′, σ₁′, σ₂′, and disease risk of the target user can be obtained as:

$${y}_{j} = {x}_{j}^{T} {σ}_{I}^{′ - 2} {{W}_{x}^{′} ( I + {σ}_{1}^{′ - 2} {W}_{x}^{′ T} {W}_{x}^{′} )}^{- 1} {W}_{y}^{′ T} \qquad ( 9 )$$

In this example, the disease risk prediction model can also be used to determine the reliability score of the disease risk value of the target user. After determining the first risk prediction parameters Wₓ′, W_{y}′, σ₁′, σ₂′, the performance parameter in the mapping relationship can be determined according to the multiple parameters, that is, the variance parameter W_{y}′CW_{y}′^{T}+σ₂′² in the p(yₙ|Xₙ), wherein C=(1+σ₁′⁻²Wₓ′^{T}Wₓ′)⁻¹. The value of the variance parameter can be calculated, and correspondingly the reliability score of the disease risk prediction model can be obtained, that is, the reliability score of the disease risk value of the target user.

Exemplarily, when it is determined that the reliability score of the disease risk prediction model is 90 points, the risk feature data of user A is input into the disease risk prediction model, and it can be obtained that the user's disease risk probability is 20%, and reliability score of the disease risk probability is 90 points. After determining the disease risk value of the target user and the reliability score of the disease risk value, the server can send them to the terminal device for display, and the target user can decide whether to perform the disease risk prediction according to the reliability score of the disease risk value presented by the terminal device.

In the disease risk prediction method provided by the exemplary embodiment of the present disclosure, by obtaining the risk feature data of the target user, a disease risk value of the target user and a reliability score of the disease risk value are determined by a disease risk prediction model based on the risk feature data. The method can more accurately determine the disease risk of the target user through the disease risk prediction model, and can obtain the reliability of the disease risk prediction model.

It should be noted that although the various steps of the methods of the present disclosure are depicted in the figures in a particular order, this does not require or imply that the steps must be performed in that particular order, or that all illustrated steps must be performed to achieve the desired the result of. Additionally or alternatively, certain steps may be omitted, multiple steps may be combined into one step for execution, and/or one step may be decomposed into multiple steps for execution, and the like.

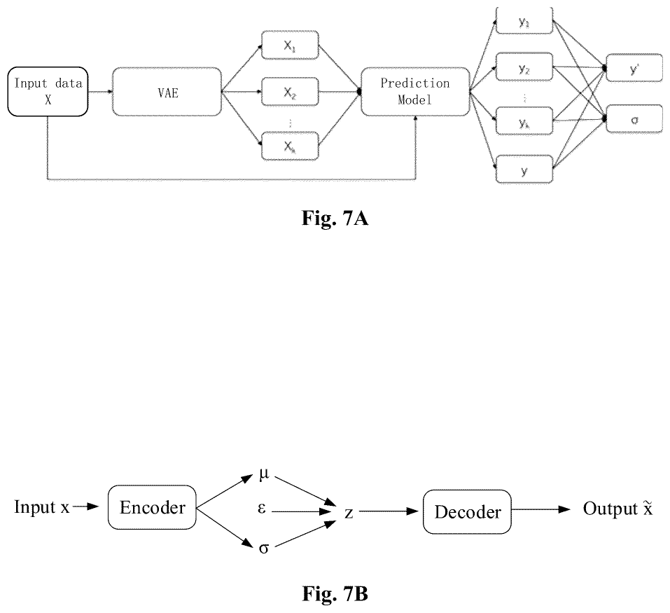

Hereinafter, a detailed example of the embodiments of the present disclosure is described with reference to FIG. 7A and FIG. 7B. FIG. 7A schematically shows a schematic diagram of the disease prediction model according to a specific embodiment of the present disclosure, and FIG. 7B schematically shows a schematic diagram of the VAE according to a specific embodiment of the present disclosure.

As shown in FIG. 7A, the example of disease prediction model generally includes two parts, the VAE module, and the prediction module.

In the example, the input data x of the disease prediction model may be (x1, x2, . . . , x11), in which xi (i=1, . . . , 11) represents the i-th risk feature shown in the above table 1. In the example, the data of category integer may be standardized to ensure that their mean is 0 and variance is 1. For the data of category boolean, the value of Y may be converted to 1 and the value of N may be converted to 0. For the data of category categorical, they may be numbered according to the category starting from 1. For example, if there are three categories in total, they are numbered 1, 2, and 3.

The VAE generally includes two parts, which as shown in FIG. 7B, are the encoder and the decoder.

The encoder according to this embodiment may include an input layer, a hidden layer and an output layer. The input layer is configured to input the feature “x” to the encoder, and the input dimension may be 11 dimensions. The hidden layer may include one or more fully connected layers to process input data through a nonlinear activation function. In this example, the present embodiment includes three fully connected layers, the nonlinear activation function is ReLU, and the number of hidden layer nodes is 128. The output layer may be configured to output two vectors, representing the mean (μ) and logarithmic variance (log σ²) of the potential distribution, and the output dimension is 16.

In the embodiment, the VAE may further include parameter reconstruction, that is, using the mean and standard deviation of the encoder output, sampling the latent variable z from the latent space using the equation z=μ+σ×ε, wherein ε follows a standard normal distribution.

The decoder according to this embodiment may include an input layer, a hidden layer and an output layer. The input layer is configured to input the latent variable z, which has a dimension of 16. The hidden layer, similar to the encoder, includes one or more fully connected layers, and nonlinear activation functions are used in between. The present invention uses three fully connected layers, the nonlinear activation function is ReLU, and the number of hidden layer nodes is 128. The output layer may be configured to output a vector with the dimension of the original feature, which is 11 in the present example (e.g., the 11 risk factors for gestational diabetes in the above table 1).

The VAE may be trained using obtained training data, and the loss function of the training may be VAE_loss=recon_loss+KL, wherein recon_loss=∥\tilde{x}−x|², and

$$KL = - log σ + \frac{{μ}^{2} + {σ}^{2}}{2} - \frac{1}{2} .$$

In the embodiment, the recon_loss may represent the reconstruction loss, which is the mean square error (MSE) to measure the difference between the decoder output \tilde{x} and the true input data x, and the KL may represent the divergence, which is used to calculate the difference between the potential distribution and the standard normal distribution. Further, the training may use the Adam optimizer and the learning rate may be set to 0.0001.

Using the trained VAE model, input x to get the corresponding μ and σ, then sample ε from the standard normal distribution, to get the latent vector z based on z=μ+σ×ε, and then use z as the input of the decoder to get \tilde{x}. Repeat this process k times to get k generated new data.

Afterwards, based on the input x and the output \tilde{x}, the risk prediction values may be obtained according to a prediction model. Any prediction model can be used to implement the functions of this module, such as neural network or logistic regression. In the example, a neural network is used for prediction, specifically a 11×64 fully connected layer, followed by a ReLu activation layer, then a 64×128 fully connected layer, a ReLu activation layer, a 128×32 fully connected layer, a ReLu activation layer, and a 32×1 fully connected layer. In the training data (x, y) of the prediction model, y is the user's disease risk size, which may be labeled by an expert, and y∈[0,10].

The loss function for the training of the prediction model may be pred_loss=|y̅−y|², which represents the mean square error between the predicted value y̅ output by the model and the true value y labeled by the expert. Further, the training may use the Adam optimizer and the learning rate may be set to 0.0001.

According this embodiment, when using this system for risk prediction, for an input x′, firstly generate k new data x₁, . . . , xₖ through the VAE, and then use the prediction model to take x′, x₁, . . . , xₖ as input respectively to obtain k+1 predicted risk values y̅′̅, y̅₁̅, . . . , y̅ₖ̅ The final prediction value is

$$\overline{y} = \frac{\overline{{y}^{′}} + \overline{{y}_{1}} + … + \overline{{y}_{k}}}{k + 1} ,$$

$$\frac{{( \overline{{y}^{′}} - \overline{y} )}^{2} + {( \overline{{y}_{1}} + \overline{y} )}^{2} + … + {( \overline{{y}_{k}} - \overline{y} )}^{2}}{k + 1} .$$

and the variance is

$${( \overline{{y}^{l}} - \overline{y} )}^{2} + {( \overline{{y}_{1}} - \overline{y} )}^{2} + … + {( \overline{Y κ} - \overline{y} )}^{2} k + 1$$

The larger the variance, the lower the reliability of the model. For prediction results with low reliability, professional evaluation can be carried out by an expert.

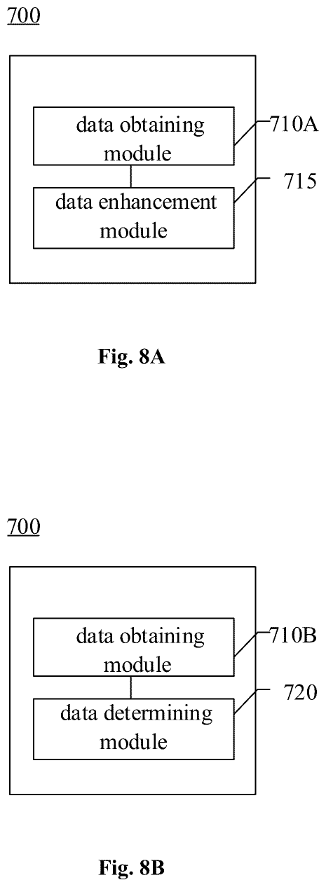

Further, in an exemplary embodiment, a disease risk prediction device is also provided. The device can be applied to a server or terminal equipment. Referring to FIG. 8A, the disease risk prediction device 700 may include a data obtaining module 710A and a data enhancement module 715.

The data obtaining module 710A is used to obtain risk feature data of a target user.

The data enhancement module 715 is used to perform data enhancement on the obtained risk feature data to obtain enhanced risk feature data.

In the embodiment, the data enhancement may be performed by using a variational autoencoder (VAE). Accordingly, the data obtaining module 710A is further used to input the obtained risk feature data to a variational autoencoder (VAE); and data enhancement module 715 is further used to obtain the enhanced risk feature data from an output of the VAE.

In further detail, the VAE may include an encoder; including an input layer, a hidden layer and an output layer, and configured to output a mean μ and logarithmic variance log(σ²) of latent distribution in response to inputting a risk feature data x; and a decoder, including an input layer, a hidden layer and an output layer, and configured to output a reconstructed value \tilde{x} in response to inputting a latent variable z, wherein the latent variable z is sampled from a latent space according to z=μ+σ×ε, wherein ε follows a standard normal distribution.

In an embodiment of the present disclosure, the VAE may be trained before using the VAE to obtain the enhanced risk feature data. In particular, training data for the VAE may be firstly obtained, and the VAE may then be trained using the training data. The loss function of the training may be VAE_loss=recon_loss+KL, wherein recon_loss=∥\tilde{x}−x∥², and

$$KL = - log σ + \frac{{μ}^{2} + {σ}^{2}}{2} - \frac{1}{2} .$$

In the embodiment, the recon_loss may represent the reconstruction loss, which is the mean square error (MSE) to measure the difference between the decoder output \tilde{x} and the true input data x, and the KL may represent the divergence, which is used to calculate the difference between the potential distribution and the standard normal distribution.

In the embodiment, referring to FIG. 8B, the disease risk prediction device 700 may include a data obtaining module 710B and a data determining module 720.

The data obtaining module 710B is used to obtain risk feature data of a target user.

In the present embodiment, the risk feature data obtained in the step S310B may be the enhanced risk feature data obtained in the step S315 of FIG. 3A. In other embodiments, the risk feature data obtained in the step S310B may also be other risk feature data, which is not limited in the present application.

In one embodiment, the enhanced risk feature data obtained in the step S315 of FIG. 3A is used as the risk feature data obtained in the step S310B, wherein the enhanced risk feature data includes original copy of the risk feature data and k copies of the output of the VAE obtained by inputting k times the risk feature data to the VAE, wherein k is an integer greater than 1. That is, the risk value x is input to the VAE for k times, and the enhanced risk feature data includes the risk value x, and all the output of the VAE of k inputs. In this embodiment, all the k+1 pieces of the enhanced risk feature data may be input to the disease risk prediction model respectively, and thus k+1 output values y̅′̅, y̅₁̅, . . . , y̅ₖ̅ may be obtained. After obtaining the k+1 output values y̅′̅, y̅₁̅, . . . , y̅ₖ̅, the disease risk value of the target user corresponding to the risk feature data x may be represented as

$$\overline{y} = \frac{\overline{{y}^{′}} + \overline{{y}_{1}} + … + \overline{{y}_{k}}}{k + 1} ,$$

and the reliability score of the disease risk value may be represented as

$$\frac{{( \overline{{y}^{′}} - \overline{y} )}^{2} + {( \overline{{y}_{1}} + \overline{y} )}^{2} + … + {( \overline{{y}_{k}} - \overline{y} )}^{2}}{k + 1} .$$

The data determining module 720 is used to determine, by the disease risk prediction model based on the risk feature data, the disease risk value of the target user and the reliability score of the disease risk value.

In an optional implementation, the data determining module 720 includes:
a first parameter determining module, used to train the disease risk prediction model to obtain a first risk prediction parameter; and
a disease risk value determining module, used to obtain the disease risk value of the target user according to the risk feature data and the first risk prediction parameter.

In an optional implementation, the first parameter determining module includes:
a second parameter determining module, used to input feature training data into the disease risk prediction model to determine a second risk prediction parameter;
a first score determining module, used to determine the reliability score of the disease risk prediction model according to the second risk prediction parameter; and
a first risk prediction parameter determining module, used to train the disease risk prediction model based on the reliability score to obtain the first risk prediction parameter.

In an optional implementation, the second parameter determining module includes:
a prediction model establishing module, used to determine a mapping relationship between the risk feature training data and the disease risk training data in a first part of the feature training data to establish the disease risk prediction model;
an objective function constructing module, used to input the risk feature training data and disease risk training data in a second part of the feature training data into the disease risk prediction model, and constructing the objective function; and
a second risk prediction parameter determining module, used to determine the second risk prediction parameter according to the objective function.

In an optional implementation, the prediction model establishing module includes:
a latent factor vector obtaining unit, used to obtain a latent factor vector corresponding to the risk feature training data;
a data distribution determining unit, used to obtain a distribution of the risk feature training data and a distribution of the disease risk training data based on the latent factor vector; and
a mapping relationship determining unit, used to establish the mapping relationship between the risk feature training data and the disease risk training data according to the distribution of the risk feature training data and the distribution of the disease risk training data.

In an optional implementation, in the mapping relationship determining unit, the mapping relationship between the risk feature training data and the disease risk training data is:

$$p ( {y}_{n} \vert {X}_{n} ) = \int p ( {y}_{n} , {Z}_{n} \vert {X}_{n} ) {dZ}_{n} = \frac{\int p ( {Z}_{n} ) \times p ( {X}_{n} \vert {Z}_{n} ) \times p ( {y}_{n} \vert {Z}_{n} ) d {Z}_{n}}{\int p ( {Z}_{n} ) \times p ( {X}_{n} \vert {Z}_{n} ) {dZ}_{n}} = N ( {y}_{n} \vert {X}_{n}^{T} v , {W}_{y} C {W}_{y}^{T} + {σ}_{2}^{2} )$$

wherein C=(1+σ₁⁻²Wₓ^{T}Wₓ)⁻¹, v=σ₁⁻²WₓCW_{y}^{T}, Xₙ is the risk feature training data of the n-th user, yₙ is the disease risk training data of the n-th user, Zₙ is the latent factor vector corresponding to the risk feature training data of the n-th user, and Wₓ, W_{y}, σ₁, σ₂ are the second risk prediction parameter in the disease risk prediction model.

In an optional implementation, the objective function is max ln p(Y|X), and wherein Y is the disease risk training data, X is the risk feature training data, and the second risk prediction parameter determining module is configured to train the risk feature training data and the disease risk training data in the second part of the feature training data using a maximum likelihood estimation algorithm, and obtain the second risk prediction parameter at a maximum probability value of the objective function.

In an optional implementation, the first score determining module includes:
a first performance parameter determining sub unit, used to determine a performance parameter corresponding to the second risk prediction parameter in the mapping relationship; and
a first score determining sub unit, used to calculate the performance parameter to obtain the reliability score of the disease risk prediction model.

In an optional implementation, the performance parameter is W_{y}CW_{y}^{T}+σ₂², wherein, C=(1+σ₁⁻²Wₓ^{T}Wₓ)⁻¹, and Wₓ, W_{y}, σ₁, σ₂ are the second risk prediction parameter in the disease risk prediction model.

In an optional implementation, the first risk prediction parameter determining module includes:
a training data obtaining sub unit, used to obtain a third part of the feature training data when the reliability score is lower than a preset threshold; and
a first risk prediction parameter determining sub unit, used to train the disease risk prediction model based on the third part of the feature training data, to obtain the first risk prediction parameter after the training is completed.

In an optional implementation, the data determining module 720 further includes:
a second risk prediction parameter determining sub unit, used to determine the performance parameter corresponding to the first risk prediction parameter in the mapping relationship; and
a second score determining sub unit, used to calculate the performance parameter to obtain the reliability score for the disease risk value.

In an optional implementation, the disease risk value determining module is configured to:

determine the disease risk value of the target user according to a relationship between the risk feature data and the first risk prediction parameter:

$${y}_{j} = {x}_{j}^{T} {σ}_{1}^{′ - 2} {{W}_{x}^{′} ( I + {σ}_{1}^{′ - 2} {W}_{x}^{′ T} {W}_{x}^{′} )}^{- 1} {W}_{y}^{′ T}$$

wherein x is the risk feature data of the target user, yⱼ is the disease risk value of the target user, and Wₓ′, W_{y}′, σ₁′, σ₂′ are the first risk prediction parameter in the disease risk prediction model.

1. A method for disease risk prediction, comprising:
obtaining risk feature data of a target user; and
performing data enhancement on the obtained risk feature data to obtain enhanced risk feature data.

2. The method for disease risk prediction according to claim 1, wherein the performing data enhancement on the obtained risk feature data to obtain enhanced risk feature data comprises:
inputting the obtained risk feature data to a variational autoencoder (VAE); and
obtaining the enhanced risk feature data from an output of the VAE.

3. The method for disease risk prediction according to claim 2, wherein the VAE comprises:
an encoder; comprising an input layer, a hidden layer and an output layer, and configured to output a mean μ and logarithmic variance log (σ²) of latent distribution in response to inputting a risk feature data x; and
a decoder, composing an input layer, a hidden layer and an output layer, and configured to output a reconstructed value \tilde{x} in response to inputting a latent variable z, wherein the latent variable z is sampled from a latent space according to z=μ+σ×ε, wherein ε follows a standard normal distribution.

4. The method for disease risk prediction according to claim 3, further comprising:
obtaining training data and training the VAE using the training data,
wherein a loss function of the training is: VAE_loss=recon_loss+KL, wherein recon_loss=∥\tilde{x}−x∥², and
$$KL = - log σ + \frac{{μ}^{2} + {σ}^{2}}{2} - \frac{1}{2} .$$

5. The method for disease risk prediction according to claim 2, further comprising:
determining, by a disease risk prediction model based on the enhanced risk feature data, a disease risk value of the target user and a reliability score of the disease risk value.

6. The method for disease risk prediction according to claim 5, wherein the enhanced risk feature data comprises original copy of the risk feature data and k copies of the output of the VAE obtained by inputting k times the risk feature data to the VAE, wherein k is an integer greater than 1, and
the determining, by the disease risk prediction model based on the enhanced risk feature data, the disease risk value of the target user and the reliability score of the disease risk value comprises:
obtaining k+1 output values y̅′̅, y̅₁̅, . . . , y̅ₖ̅ by inputting each copy of the enhanced risk feature data into the disease risk prediction model;
determining the disease risk value of the target user as
$$\overline{y} = \frac{\overline{{y}^{′}} + \overline{{y}_{1}} + … + \overline{{y}_{k}}}{k + 1} ;$$
and
determining the reliability score of the disease risk value as
$$\frac{{( \overline{{y}^{′}} - \overline{y} )}^{2} + {( \overline{{y}_{1}} + \overline{y} )}^{2} + … + {( \overline{{y}_{k}} - \overline{y} )}^{2}}{k + 1} .$$

7. The method for disease risk prediction according to claim 5, wherein the determining, by the disease risk prediction model based on the risk feature data, the disease risk value of the target user comprises:
the disease risk prediction model comprising a first risk prediction parameter; and
obtaining the disease risk value of the target user based on the risk feature data and the first risk prediction parameter.

8. The method for disease risk prediction according to claim 7, comprising:
training the disease risk prediction model to obtain the first risk prediction parameter,
wherein the training the disease risk prediction model to obtain the first risk prediction parameter comprises:
inputting feature training data into the disease risk prediction model to determine a second risk prediction parameter;
determining a reliability score of the disease risk prediction model according to the second risk prediction parameter; and
training the disease risk prediction model based on the reliability score to obtain the first risk prediction parameter.

9. The method for disease risk prediction according to claim 8, wherein the feature training data comprises risk feature training data and disease risk training data, and
the inputting the feature training data into the disease risk prediction model to determine the second risk prediction parameter comprises:
determining a mapping relationship between the risk feature training data and the disease risk training data in a first part of the feature training data to establish the disease risk prediction model;
inputting the risk feature training data and disease risk training data in a second part of the feature training data into the disease risk prediction model, and constructing a objective function; and
determining the second risk prediction parameter according to the objective function.

10. The method for disease risk prediction according to claim 9, wherein the determining the mapping relationship between the risk feature training data and the disease risk training data in the first part of the feature training data comprises:
obtaining a latent factor vector corresponding to the risk feature training data;
obtaining a distribution of the risk feature training data and a distribution of the disease risk training data based on the latent factor vector; and
establishing the mapping relationship between the risk feature training data and the disease risk training data according to the distribution of the risk feature training data and the distribution of the disease risk training data.

11. The method for disease risk prediction according to claim 10, wherein the mapping relationship between the risk feature training data and the disease risk training data is:
$$p ( {y}_{n} \vert {X}_{n} ) = \int p ( {y}_{n} , {Z}_{n} \vert {X}_{n} ) {dZ}_{n} = \frac{\int p ( {Z}_{n} ) \times p ( {X}_{n} \vert {Z}_{n} ) \times p ( {y}_{n} \vert {Z}_{n} ) d {Z}_{n}}{\int p ( {Z}_{n} ) \times p ( {X}_{n} \vert {Z}_{n} ) {dZ}_{n}} = N ( {y}_{n} \vert {X}_{n}^{T} v , {W}_{y} C {W}_{y}^{T} + {σ}_{2}^{2} )$$
wherein C=(1+σ₁⁻²Wₓ^{T}Wₓ)⁻¹, v=σ₁⁻²WₓCW_{y}^{T}, Xₙ is the risk feature training data of the n-th user, yₙ is the disease risk training data of the n-th user, Zₙ is the latent factor vector corresponding to the risk feature training data of the n-th user, and Wₓ, W_{y}, σ₁, σ₂ are the second risk prediction parameter in the disease risk prediction model.

12. The method for disease risk prediction according to claim 9, wherein the objective function is max ln p(Y|X), and wherein Y is the disease risk training data, X is the risk feature training data, and
wherein the determining the second risk prediction parameter according to the objective function comprises:
training the risk feature training data and the disease risk training data in the second part of the feature training data using a maximum likelihood estimation algorithm, and obtaining the second risk prediction parameter at a maximum probability value of the objective function.

13. The method for disease risk prediction according to claim 9, wherein the determining the reliability score of the disease risk prediction model according to the second risk prediction parameter comprises:
determining a performance parameter corresponding to the second risk prediction parameter in the mapping relationship; and
calculating the performance parameter to obtain the reliability score of the disease risk prediction model.

14. The method for disease risk prediction according to claim 13, wherein the performance parameter is W_{y}CW_{y}^{T}+σ₂², wherein, C=(1+σ₁⁻²Wₓ^{T}Wₓ)⁻¹, and Wₓ, W_{y}, σ₁, σ₂ are the second risk prediction parameter in the disease risk prediction model.

15. The method for disease risk prediction according to claim 13, wherein the training the disease risk prediction model based on the reliability score to obtain the first risk prediction parameter comprises:
acquiring a third part of the feature training data when the reliability score is lower than a preset threshold; and
training the disease risk prediction model based on the third part of the feature training data, to obtain the first risk prediction parameter after the training is completed.

16. The method for disease risk prediction according to claim 15, wherein the determining reliability score of the disease risk value by the disease risk prediction model comprises:
determining the performance parameter corresponding to the first risk prediction parameter in the mapping relationship; and
calculating the performance parameter to obtain the reliability score for the disease risk value.

17. The method for disease risk prediction according to claim 7, wherein the obtaining the disease risk value of the target user based on the risk feature data and the first risk prediction parameter comprises:
determining the disease risk value of the target user according to a relationship between the risk feature data and the first risk prediction parameter:
$${y}_{j} = {x}_{j}^{T} {σ}_{1}^{′ - 2} {{W}_{x}^{′} ( I + {σ}_{1}^{′ - 2} {W}_{x}^{′ T} {W}_{x}^{′} )}^{- 1} {W}_{y}^{′ T}$$
wherein xⱼ is the risk feature data of the target user, yⱼ is the disease risk value of the target user, and Wₓ′, W_{y}′, σ₁′, σ₂′ are the first risk prediction parameter in the disease risk prediction model.

18. A non-transient computer-readable storage medium stored thereon with a computer program, which when being executed by a processor, implement the method according to claim 1.

19. An electronic device, comprising:
a processor; and
a non-transient memory, configured to store instructions executable by the processor,
wherein the processor is configured to execute the executable instructions to:
obtain risk feature data of a target user; and
perform data enhancement on the obtained risk feature data to obtain enhanced risk feature data.

20. The electronic device according to claim 19, wherein the processor is further configured to:
determine, by a disease risk prediction model based on the risk feature data, a disease risk value of the target user and a reliability score of the disease risk value.
